(19) United States
(12) Patent Application Publication
(10) Pub. No.: US 2017/0197158 A1
(43) Pub. Date: Jul. 13, 2017

(54) PARTICLE SETTLING DEVICES

(71) Applicant: SUDHIN BIOPHARMA, Superior, CO (US)

(72) Inventor: Dhinakar S. KOMPALA, Superior, CO (US)

(21) Appl. No.: 15/324,062

(22) PCT Filed: Jul. 9, 2015
(86) PCT No.: PCT/US15/39723

§ 371 Date: Jan. 5, 2017

Related U.S. Application Data
(60) Provisional application No. 62/022,276, filed on Jul. 9, 2014.
(60) Provisional application No. 62/037,513, filed on Aug. 14, 2014.

(51) Int. Cl. B01D 21/26; B04C 5/103; C02F 1/38; C12N 5/04; C12N 1/18; C12N 1/02; C12N 5/00; C12N 7/00; B01D 21/00; C07K 1/14

(52) U.S. Cl. CPC B01D 21/265; B01D 21/0054; B04C 5/103; C02F 1/385; C07K 1/14; C12N 1/18; C12N 1/02; C12N 5/00; C12N 7/00; C12N 5/04

(57) ABSTRACT

Settling devices for separating millimeter or micron sized particles from a bulk fluid with applications in numerous fields, such as biological (microbial, mammalian, insect, plant, or algal) cell cultures, solid catalyst particle separation from a liquid or gas and waste water treatment.

### FIELD OF THE INVENTION

This disclosure provides a particle settling device with enhanced settling on the multilayered inclined curved surfaces.

### DESCRIPTION OF RELATED ART

Separating and collecting biological proteins, polypeptides or hormones secreted from suspension cultures of recombinant microbial or mammalian cells is a particularly challenging task. Most common methods of producing biological proteins in recombinant mammalian and microbial cells rely on fed-batch cultures, wherein cells are grown to high cell densities and then typically exposed to an induction medium or inducer to trigger the production of proteins. If the desired proteins are secreted out of the cells, it is more profitable to switch from a fed-batch culture to a continuous perfusion culture, which can maintain high cell density and high productivity over a much longer duration of culture. During continuous perfusion cultures, live and productive cells are retained or recycled back to the bioreactor while the secreted proteins are continuously harvested from the bioreactor for downstream purification processes.

Some key advantages of continuous perfusion cultures over fed-batch cultures are: (1) the secreted protein products are continuously removed from the bioreactor, without subjecting these products to potential degradation by proteolytic and/or glycolytic enzymes released into the culture medium from dead cells, (2) live and productive cells are retained or recycled back to achieve high cell densities in continuous perfusion bioreactors, where they continue to produce valuable proteins inside the controlled bioreactor environment for much longer culture duration, rather than being removed from the bioreactor at the end of each fed-batch culture (3) the perfusion bioreactor environment can be maintained much closer to steady state conditions (thereby maintaining a constant product quality) with the continuous addition of fresh nutrient media and removal of waste products along with the harvested protein products, unlike the dynamically changing concentrations of nutrients and waste products in fed-batch culture, and (4) with a subset of cell retention devices, smaller dead or dying cells can be selectively removed from the perfusion bioreactor before these cells lyse and release their intracellular enzymes, thereby maintaining a high viability fraction of cells and high quality of the secreted protein products as they are harvested.

Many cell retention devices have been developed in the mammalian cell culture industry, such as the internal spin filter devices (Himmelfarb et al., Science 164: 555-557, 1969), external filtration modules (Brennan et al., Biotechnol. Techniques, 1 (3): 169-174, 1987), hollow fiber modules (Knazek et al., Science, 178: 65-67, 1972), gravitational settling in a cyclone (Kitano et al., Appli. Microbiol. Biotechnol. 24, 282-286, 1986), inclined settlers (Batt et al., Biotechnology Progress, 6:458-464, 1990), continuous centrifugation (Johnson et al., Biotechnology Progress, 12, 855-864, 1999), and acoustic filtering (Gorenflo et al., Biotechnology Progress, 19, 30-36, 2003). The cyclones were found to be incapable of producing enough centrifugal force for sufficient cell separation at the device sizes and harvest flow rates used in the mammalian cell culture experiments (Kitano et al., 1986) and mammalian cells are seriously damaged at higher flow rates (and centrifugal forces) necessary for efficient cell separation (Elsayed, et al., Eng. Life Sci., 6: 347-354, 2006). While most of the other devices adequately retain all mammalian cells from the harvest, these devices are unable to separate dead cells from the live cells desired in the bioreactor. Consequently, dead cells keep accumulating inside the perfusion bioreactor and the membrane filters get clogged, necessitating the termination of the continuous perfusion bioreactor, typically in less than a week.

Among all the cell retention devices available today, only the inclined settlers (Batt et al., 1990, supra and Searles et al., Biotechnology Progress, 10: 198-206, 1994) enable selective removal of smaller dead cells and cell debris in the overflow or harvest stream, while bigger, live and productive mammalian cells are continually recycled via the underflow back to the perfusion bioreactor. Therefore, operation of the perfusion bioreactor may continue indefinitely at high viability and high cell densities while the protein product is continuously harvested from the top of the inclined settler.

The inclined settler has previously been scaled up as multi-plate or lamellar settlers (Probstein, R. F., U.S. Pat. No. 4,151,084, April 1979) and used extensively in several large-scale industrial processes such as wastewater treatment, potable water clarification, metal finishing, mining and catalyst recycling (e.g. Odueyngbo et al., U.S. Pat. No. 7,078,439, July 2006). Citing the first demonstration of a single plate inclined settler (Batt et al., 1990) to enhance productivity of secreted proteins in mammalian cell culture applications, a multi-plate or lamellar settler device has been patented for the scale up of inclined settlers for use in hybridoma cell culture (Thompson and Wilson, U.S. Pat. No. 5,817,505, October 1998). Such lamellar inclined settler devices have been used to operate continuous perfusion bioreactors at high bioreactor productivity (due to high cell density) and high viability (>90%) for long durations (e.g. several months without any need to terminate the perfusion culture).

None of these cell retention devices have been demonstrated for harvesting secreted protein products in perfusion bioreactor cultures of the smaller, and hence more challenging, microbial cells. Lamellar settlers have been tested with yeast cells to investigate cell settling with limited success (Bungay and Millspaugh, Biotechnology and Bioengineering, 23:640-641, 1984). Hydrocyclones have been tested in yeast suspensions, mainly to separate the yeast cells from beer, again with only limited success (Yuan et al., Bioseparation, 6: 159-163, 1996, Cilliers and Harrison, Chemical Engineering Journal, 65: 21-26, 1997).

A modified cyclone with a spiral vertical plate inside the cyclone was proposed to improve the separation efficiency in wastewater treatment (Boldyrev V V, Davydov E I, settling tanks, as described in Russian Patent No. 2,182,508) and an earlier description of this arrangement has been described for the decantation of solids in liquid suspension (U.S. Pat. No. 4,048,069, September 1977). The modified cyclone disclosed in this Russian patent includes a spiral wound plate housed in a vertical cylindrical barrel with a conical bottom. A slit is provided along the entire height of a central waste water inlet tube, which is plugged at the bottom in order to channel waste water from the inlet tube into the vertical spiral wound plate. The spiral starts at the central tube and ends at the wall of the cylindrical housing, forming a channel through which particle-laden waste water flows. The particles settle in the vertical sedimentation column of the spiral channel. The height of the settler zone is the vertical height of the spiral plate and the width of the channel is formed by the walls of the spiral wound plate, which is held constant throughout its length. A pipe for removing the purified water is installed at the upper part of the cylindrical body. A conduit for removing sediment is installed at the bottom of the conical bottom portion. In operation, waste water enters through the central tube and enters the spiral zone through the slit or opening. The spiral channel serves to increase the flow path and hence increase the residence time of liquid in the settler. The spiral also serves to increase the contact (wall) area for the fluid. The main driving force in clarification is gravity acting on the particles of the suspension, as the suspension goes around the spiral-wound vertical sedimentation column. The slurry that is left on the wall of the spiral or in the channel, falls into the conical bottom of the settler, and is removed periodically from the settler. Purified water is drawn from a pipe on the side of cylindrical housing near the top. As described in the Russian patent document, the flow pattern of the waste water-containing solids is reversed from the typical flow pattern of a common cyclone, as the dirty water enters at the center, via the central tube and enters into the spiral channel through the slits, and the purified water is removed from the periphery or outside of the vertical cylindrical body via a purified water pipe. This modified and flow-reversed cyclone device has not been proposed for, or applied to any fields other than waste water treatment.

Thus, a particle settling device that can leverage centrifugal forces and gravitational forces on particles in liquid suspension in a relatively small space is desired.

### SUMMARY OF THE INVENTION

This disclosure provides particle settling devices with enhanced settling on multilayered, inclined surfaces that may be attached to a plurality of vertical cylindrical plates. The particle separation devices of this disclosure may be used in numerous applications, and represent a large improvement over the prior art separation devices. The devices include a spiral conical surface, or several inclined plates approximating an angled conical surface connected to the bottom of a spiral. The numerous, layered inclined enhance the settling efficiency of the particles from the bulk fluid moving either downward or upward inside a conical cyclone assembly in which the liquid volume moves progressively from the periphery of the conical spiral to the center of the settler device.

In one or more embodiments, the devices of this disclosure include a cyclone (often referred to as a “hydrocyclone”) housing, a spiral vertical plate positioned inside the cyclone housing, the spiral vertical plate joined at its bottom with a spiral conical surface tapering down to an opening. Notably, there is no plug or other impediment preventing the flow of liquid or suspended particles from the spiral vertical plates or spiral conical surfaces toward the opening. The spiral conical surface forms lamellar inclined settler plates in a conical geometry.

In related embodiments, the devices of the invention include a cyclone housing, a spiral vertical plate positioned inside the cyclone housing, the spiral vertical plate joined at its bottom with a spiral conical settling surface tapering down to an opening. In this embodiment, the vertical spiral plate has a decreasing height towards the center of the device, and constant spacing between the successive spiral rings. The spiral conical settling surfaces at the bottom of a spiral vertical plate have increasing lengths to match the decreasing height of the vertical spiral plate and extend to approximately the center of the settler device. Similarly, there is no plug or other impediment preventing the flow of liquid or suspended particles from the spiral vertical plates or spiral conical surfaces toward the opening.

In all of the embodiments described above, attaching the spiral vertical plates to the spiral conical settling surfaces can be accomplished by welding or otherwise joining (i.e., gluing or other adhesives, bonding, ultrasonic welding, clamping, or the like) curved angular plates at a fixed inclination to the circular bottom edge of the spiral vertical plate.

In all of the embodiments described above, the spiral conical surface can be tightly fitted to obtain a continuous conical spiral surface. Alternatively, small gaps between the spiral conical surfaces are acceptable for a discontinuous conical spiral surface, provided the gaps in the surface are staggered between successive conical spirals.

In all of the embodiments described above, the angle of inclination for the conical spiral surfaces can be between 30 degrees and 60 degrees from the vertical. In certain embodiments, the angle of inclination for the conical spiral surfaces is about 45 degrees from the vertical. For stickier particles (typically mammalian cells), the angle of inclination is preferably closer to the vertical (i.e., about 30 degrees from the vertical. For non-sticky solid particles (for example, catalyst particles), the angle of inclination can be further from the vertical (preferably, about 60 degrees from vertical).

In other embodiments, the settler device of this disclosure includes a cyclone housing that encloses a series of stacked cones positioned inside the cyclone housing, tapering down to a central opening, with no vertical plates. The cones of this embodiment are supported in the stack, one above the other, by vertical supports that maintain a distance (or channel width) between the successive cones in the stack. In certain embodiments, the vertical supports comprise three or more projections attached to the upper and/or lower surface of one or more of the cones to position successive cones at a desired distance (the desired channel width) apart. As in the previous embodiments, there is no plug or other impediment preventing the flow of liquid or suspended particles from the stacked conical surfaces toward the central opening.

In all of the embodiments of the settler devices of this disclosure, the components of the settler devices may be composed of a metal and/or a plastic. In certain embodiments, the components of the settler devices are composed of stainless steel. In specific embodiments these settler devices are composed entirely of stainless steel. In specific embodiments including a spiral vertical plate, and the spiral conical surface and the spiral vertical plate are metals joined by welding. In other embodiments, these settler devices are composed entirely of plastic. In all of these embodiments, the surface of the cyclone housing, the spiral vertical plate or the conical surfaces may be completely or partially coated with a non-sticky plastic or silicone or the metals (especially stainless steel) may be electropolished to provide a smooth surface.

All of the embodiments of the settler devices of this disclosure may include a closure or lid over at least a portion of the cyclone housing at an end of the cyclone housing opposite the first opening. In all of these embodiments, the closure or lid may also include an outlet or port for removing liquids or entering liquids into the settler device. In all of these embodiments, the opening and the additional ports or outlets in the cylindrical housing and/or the lid are in liquid communication with the outside and the inside of the cyclone housing to allow the passage of liquids into and/or out of the cyclone housing of the settler device, and in each instance of such opening or inlet/outlet, these passage ways into and out of the cyclone housing may include valves or other mechanisms that can be opened or closed to stop or restrict the flow of liquids into or out of the settler devices of this disclosure.

An important factor causing particle separation in the settler devices of this disclosure is the enhanced sedimentation on the inclined surfaces, which has been successfully demonstrated by Boycott (Nature, 104: 532, 1920) with blood cells and Batt et al. (1990) with hybridoma cells producing monoclonal antibodies. Minor additional factors enhancing the particle separation include the centrifugal force on the particles during their travel through the spiral channel and the settling due to gravity in the vertical sedimentation columns. While lamellar plates have been used to scale up inclined plate settlers by each dimension independently, i.e. increasing the length, or the width or the number of plates stacked on top of the each plate, the spiral conical settling zone can be scaled up in three dimensions simultaneously by simply increasing the horizontal radius of this device. As the horizontal radius of the device increases, the number of vertical and conical surfaces can be proportionally increased by keeping a constant distance (or channel width) between the successive spirals. The particle separation efficiency is directly proportional to the total projected horizontal area of the inclined settling surfaces. With an increase in device radius, the projected horizontal area increases proportional to the square of the radius, and the number of feasible spiral cones at a channel width also increases with the radius, resulting in a three dimensional scale up in the total projected area (i.e. proportional to the cube of radius) by simply increasing the radius.

Thus, another aspect of this disclosure provides a method of settling particles in a liquid suspension including introducing a liquid suspension into a particle settling device of this disclosure and collecting particles from a first opening in the cyclone housing and collecting a clarified liquid from another opening in the settling device. In certain embodiments of this method, the clarified liquid is collected from an opening in a closure that covers at least a portion of the cyclone housing at an end of the cyclone housing opposite the first opening. In certain embodiments, clarified liquid is collected from at least one additional opening in the cyclone housing, which opening is configured to open from a side of the cyclone housing.

In certain embodiments of these methods, the liquid suspension may include a recombinant cell suspension, an alcoholic fermentation, a suspension of solid catalyst particles, a municipal waste water, industrial waste water. In certain embodiments of these methods, the liquid suspension may include mammalian cells, bacterial cells, yeast cells, plant cells, and/or insect cells. In certain embodiments of these methods, the liquid suspension may include biodiesel-producing algae cells, mammalian and/or murine hybridoma cells, and yeast in beer. In certain embodiments of these methods, the liquid suspension may include recombinant microbial cells selected from Pichia pastoris, Saccharomyces cerevisiae, Kluyveromyces lactis, Aspergillus niger, Escherichia coli, and Bacillus subtilis.

In certain embodiments of these methods, the step of introducing a liquid suspension into the settler device includes directing a liquid suspension from a plastic bioreactor bag into the particle settling device.

In certain embodiments of these methods, the clarified liquid collected from the settler device includes at least one of biological molecules, organic or inorganic compounds, chemical reactants, and chemical reaction products. In certain embodiments of these methods, the clarified liquid collected from the settler device includes at least one of hydrocarbons, polypeptides, proteins, alcohols, fatty acids, hormones, carbohydrates, antibodies, isoprenoids, biodiesel, and beer. In certain embodiments of these methods, the clarified liquid collected from the settler device includes at least one of insulin or its analogs, monoclonal antibodies, growth factors, sub-unit vaccines, viruses, virus-like particles, colony stimulating factors and erythropoietin (EPO).

The preceding is a simplified summary of the disclosure to provide an understanding of some aspects of the settler devices of this disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

### BRIEF DESCRIPTION OF FIGURES

FIG. 1A shows a cross sectional view through the side of one embodiment of a conical spiral settler device of this disclosure. FIG. 1B is a cross sectional view through the top of the conical spiral settler device of FIG. 1A, 3 showing a top view of spiral plates inside a cyclone housing

FIG. 2 shows a cross sectional view through another embodiment of the settler device of this disclosure, without the conical spiral surface.

FIG. 3 shows a cross sectional view of an alternate configuration of a spiral conical surface, with extensions to the conical settler surface to ensure the upward flow of cell culture broth through all the conical spiral and vertical sedimentation chambers within a settler device of this disclosure

FIG. 4 shows a cross sectional view through the side of one embodiment of a conical settler device of this disclosure.

FIG. 5, FIG. 6 and FIG. 7 show schematic diagrams of different liquid flow patterns through a bioreactor of this disclosure.

### DESCRIPTION OF EMBODIMENTS

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The phrases “at least one,” “one or more,” and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

The transitional term “comprising” is synonymous with “including,” “containing,” or “characterized by,” is inclusive or open-ended and does not exclude additional, unrecited elements or method steps.

The transitional phrase “consisting of” excludes any element, step, or ingredient not specified in the claim, but does not exclude additional components or steps that are unrelated to the invention such as impurities ordinarily associated therewith.

The transitional phrase “consisting essentially of” limits the scope of a claim to the specified materials or steps and those that do not materially affect the basic and novel characteristic(s) of the claimed invention.

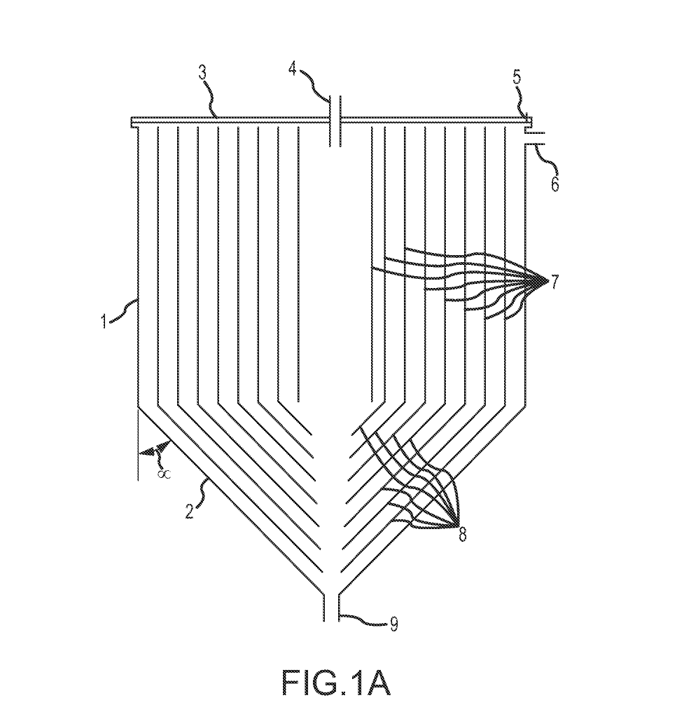

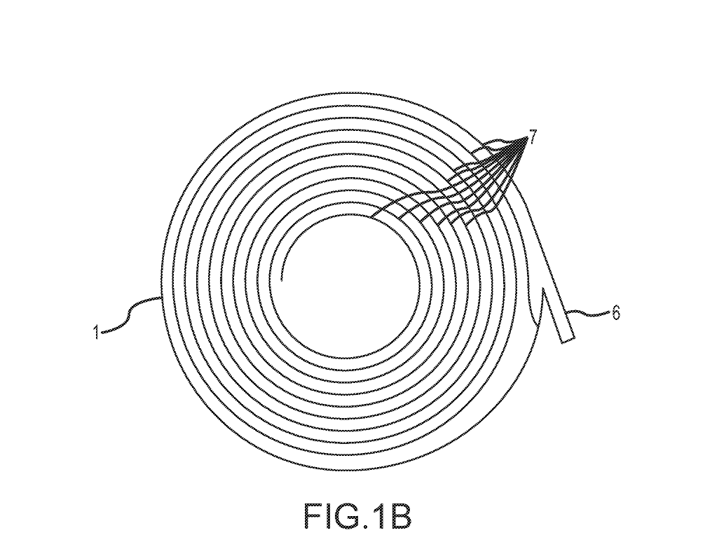

In one embodiment, depicted in FIGS. 1A and 1B, a settler device of this disclosure includes a cyclone housing (1) enclosing spiral vertical plate (7), the spiral vertical plate (7) joined at one end with a conical surface (8) tapering down to an opening (9). As depicted in FIG. 1B, the spiral vertical plate (7) is supported within the cyclone housing by attachment to the cyclone housing (1). Optionally, the spiral vertical plate (7) may also include one or more supporting attachments to a top plate (3).

Opening (9) is of sufficient diameter to allow removal of settled cells or particles. Preferably, there is a constant spacing between successive rings of the spiral vertical plate (7). The conical surface (8) joined to the spiral vertical plate (7) may be formed as a single continuous spiral surface, or individual angled plates, and acts as a lamellar inclined settler plate, in a conical geometry.

The cyclone housing (1), may optionally include a means to control the temperature of the settler device, such as a temperature control jacket or reservoir for cooling or heating fluids to be circulated around all or part of the cyclone housing (1).

The conical bottom portion (2) of the cyclone housing (1) extends from a vertical surface of the cyclone housing (1) to the opening (9) and is preferably positioned at an angle a from the vertical that matches the angle of at least one conical surface (8).

Top plate (3), which may function as a lid to the cyclone housing, may be optionally attached to the top of the cyclone housing (1) by at least one screw (5). The top plate (3) may optionally be secured in place over the cyclone housing (1) over an o-ring (not shown). Central top port (4) may act as an inlet or outlet port for liquid and/or particles entering or exiting the settler device through the top plate (3). Similarly, one or more tangential ports (6) located in the cyclone housing (1) may also act as an inlet or outlet port for liquid and/or particles entering or exiting the settler device through the cyclone housing (1). These one or more tangential ports (6) may be positioned in the cyclone housing (1) at any position between the opening (9) and the top plate (3). The tangential ports (6) may each be dedicated inlet ports, dedicated outlet ports, or dual function inlet/outlet ports, for the transfer of liquid and/or particles into or out of the settler device. As noted above, there is no plug or other impediment preventing the flow of liquid or suspended particles from the spiral vertical plate (7) or the conical surfaces (8), toward the opening (9).

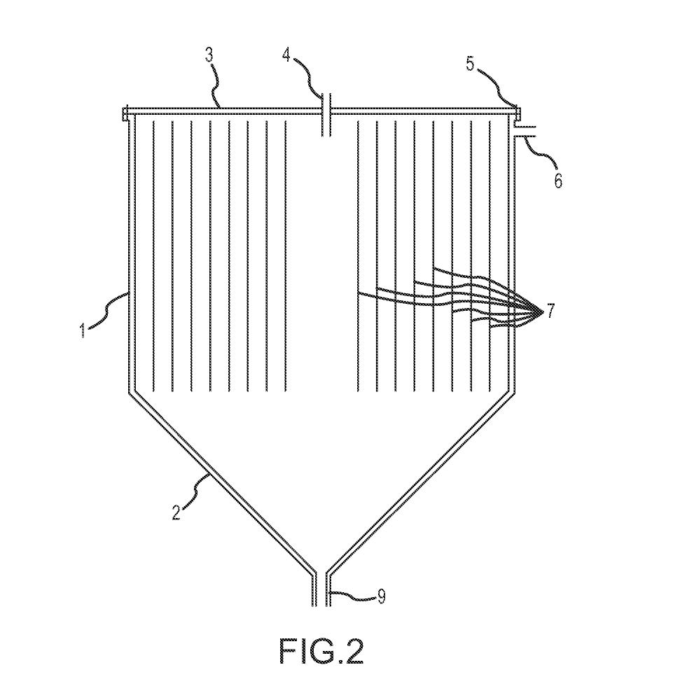

A simpler, modified version of the settler device of this embodiment is depicted in FIG. 2. In this embodiment, the conical surface (FIG. 1, reference number 8) is omitted. This modified version also works well for many separation applications, albeit with reduced efficiency of particle separation compared to the settler device depicted in FIG. 1, due to the synergistic effects of centrifugal forces acting on particles in this settler device, when a liquid suspension of particles is introduced through a tangential port (6) near the top of cyclone housing (1) proximate the top plate (3) and the increased residence time of particles in the vertical sedimentation channel of the vertical conical plate (7).

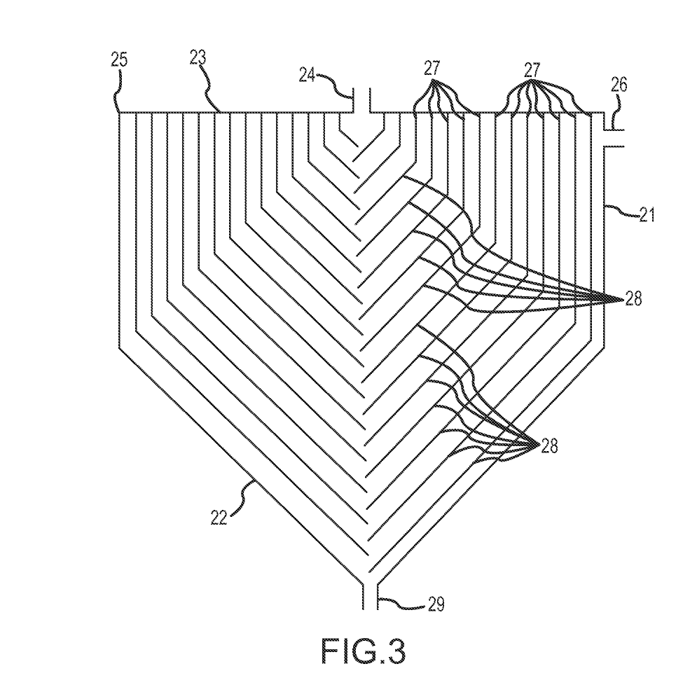

Another embodiment of the settler device of this disclosure is depicted in FIG. 3. This embodiment is particularly useful for smaller scale particle separation applications, such as particle settlers for use with a plastic bag bioreactor using only two vertical ports. In this embodiment, the settler device of this disclosure includes a cyclone housing (21) enclosing spiral vertical plate (27), the spiral vertical plate (27) joined at one end with a conical surface (28) tapering down to an opening (29). The spiral vertical plate (27) is supported within the cyclone housing by attachment to the cyclone housing (not shown). Optionally, the spiral vertical plate (27) may also include one or more supporting attachments to a top plate (23). The spiral vertical plate (27) is formed with progressively longer vertical spirals, moving from the center of the settler device of this embodiment towards the cyclone housing (21). The conical surfaces (28) joining one end of the spiral vertical plate (27) are formed in increasingly longer lengths to extend from the joined end of the spiral vertical plates (27) to a position proximate the center of the settler device, in order to direct cells or particles towards the opening (29). As depicted in FIG. 3, the end of the conical surfaces (28) opposite the end joining the spiral vertical plate (27) may extend beyond the center of the settler device to partially overlap successive conical surfaces (28). As noted above, there is no plug or other impediment preventing the flow of liquid or suspended particles from the spiral vertical plate (27) or the conical surfaces (28), toward the opening (29). As with the embodiment depicted in FIG. 1, there is preferably a constant spacing between successive rings of the spiral vertical plate (27), and the conical surface (28) joined to the spiral vertical plate (27) may be formed as a single continuous spiral surface, or individual angled plates. For use in small scale, bioreactor or biobag separation applications, opening (29) may function for inlet of cell culture broth as well as recycling settled cells or particles back to a biobag or bioreactor.

The cyclone housing (21), including the conical bottom portion (22), of this embodiment may also include a means to control the temperature of the settler device.

Top plate (23) is optionally attached to the top of the cyclone housing (21) by at least one screw (25), and may be secured in place over the cyclone housing (21) over an o-ring (not shown). Central top port (24) may act as an inlet or outlet port for liquid and/or particles entering or exiting the settler device through the top plate (23). In this embodiment, central top port 24 is particularly useful for removing clarified cell culture liquid. Similarly, one or more optional tangential ports (26) located in the cyclone housing (21) may also act as an inlet or outlet port for liquid and/or particles entering or exiting the settler device through the cyclone housing (21). These one or more optional tangential ports (26) may be positioned in the cyclone housing (21) at any position between the opening (29) and the top plate (23). The optional tangential ports (26) may each be dedicated inlet ports, dedicated outlet ports, or dual function inlet/outlet ports, for the transfer of liquid and/or particles into or out of the settler device. Such optional tangential port (26) located in the cyclone housing (21) proximate the top plate (23) is typically not needed in small scale, bioreactor or biobag separation applications, but may be useful for faster filling of the settler device with cell culture liquids before priming a pump in liquid communication with the central top port (24), as described below. If the optional tangential inlet port (26) is not used, the cell culture broth can be sucked up through opening (29) by a peristaltic pump in fluid communication with the central top port (24), as described below.

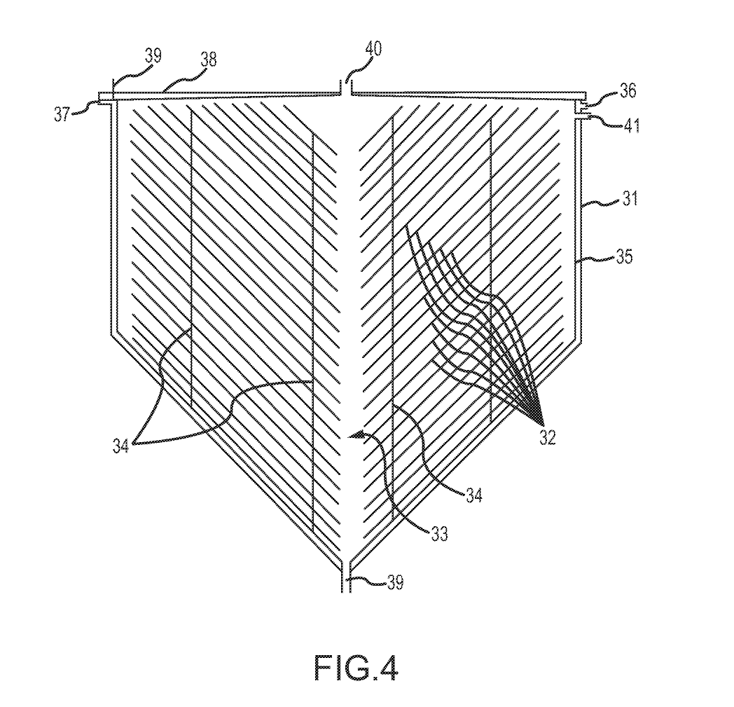

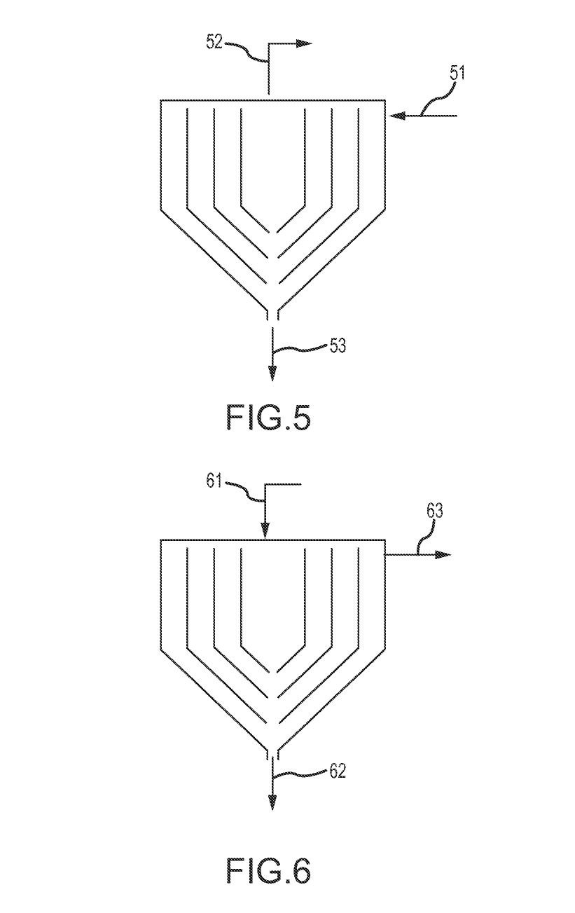

Another embodiment of the settler device of this disclosure is depicted in FIG. 4. This embodiment is particularly useful for separation applications in which the particle settling device requires regular or continual service, such as disassembly and cleaning of the conical settling surfaces within the settler device. In this embodiment, the settler device of this disclosure includes a cyclone housing (31) enclosing a stack of two or more stacked cones (32), each having a central opening (33), the cyclone housing (31) tapering down to an opening (39). The stacked cones (32) comprise at least three vertical supports (34) supporting each cone (32) above the next successive cone (32) in the stack. In preferred embodiments, the vertical supports (34) are preferably placed at a constant distance and are formed at an equal length to hold each successive cone (32) in the stack at an equal spacing between all of the cones (32) in the stack. There should be at least three vertical supports for each cone (32) to properly support each cone, but each cone (32) may comprise more than three vertical supports as needed to adequately or properly support the cone (32). However, each vertical support represents an impediment to settled particles or cells sliding down the surface of the cone (32) towards the central opening (33).

The vertical supports (34) may be attached to the top of each cone (32), thereby supporting the next successive cone (32) in the stack. Alternatively or additionally, the vertical supports (34) may be attached to the bottom of each cone (32), thereby supporting the cone (32) above the next successive cone (32) in the stack.

As noted above, there is no plug or other impediment preventing the flow of liquid or suspended particles from the central opening (33) in each cone (32) toward the opening (39).

As depicted in FIG. 4, the cyclone housing (31), may include a means to control the temperature of the settler device, such as reservoir (35) for cooling or heating fluids to be circulated around all or part of the cyclone housing (31). Ports (36, 37) may be inlet or outlet ports for the circulation of heating or cooling fluids through the reservoir (35).

Top plate (38) is optionally attached to the top of the cyclone housing (31) by at least one screw (39), and may be secured in place over the cyclone housing (31) over an o-ring (not shown). Central top port (40) may act as an inlet or outlet port for liquid and/or particles entering or exiting the settler device through the top plate (38). Central top port (40) is particularly useful for removing clarified cell culture liquid. Similarly, one or more optional tangential ports (41) located in the cyclone housing (31) may also act as an inlet or outlet port for liquid and/or particles entering or exiting the settler device through the cyclone housing (31). These one or more optional tangential ports (41) may be positioned in the cyclone housing (31) at any position between the opening (39) and the top plate (38). The optional tangential ports (41) may each be dedicated inlet ports, dedicated outlet ports, or dual function inlet/outlet ports, for the transfer of liquid and/or particles into or out of the settler device.

In each of the embodiments of this disclosure, the number of spirals or cones typically range from about 3 to about 30 or more, depending on the radius of the device. In each of the embodiments of this disclosure, the channel width (i.e., the distance between each successive spiral or each successive conical cone) can range between about 1 mm and about 50 mm. For larger flow rates, device sizes, and dense fluids, the larger channel width will be preferable to minimize the pressure drop or friction. A smaller channel width can increase the number of spirals or cones that can fit inside a given radius of the device. Smaller channel widths are, however, more prone to clogging by dense packing of the settled or settling particles. The thickness of spiral or cone material should be as small as possible to maintain the rigidity of shape while minimizing the weight of the spiral or cones supported inside the cyclone housing.

The radius and size of these settler devices can be scaled up easily in three dimensions, as much as needed for large-scale/large-volume processes. However, the scale up of these devices needs to be carried out empirically, as theoretical development of predictive equations is not yet available, as they were for lamellar settlers (Batt et al. 1990). These settler devices can be scaled up or down to suit the separation needs of different industries or applications or sizes as the separation surface is scaled up or down approximately in three dimensions, compared to the more typical one- or two-dimensional scaling of previous settling devices.

In each of the embodiments of this disclosure, the angle of inclination of the surfaces of the conical surfaces or the stacked cones can also be between 30 degrees and 60 degrees from the vertical. In certain embodiments, the angle of inclination for the surfaces of the conical surfaces or stacked cones is about 45 degrees from the vertical. As described above, for the separation of stickier particles (typically mammalian cells), the angle of inclination is preferably closer to the vertical (i.e., about 30 degrees from the vertical). For less-sticky solid particles (for example, catalyst particles), the angle of inclination can be further from the vertical (preferably, about 60 degrees from vertical).

The material of construction of any of the settler devices of this disclosure can be stainless steel (especially stainless steel 316), or similar materials used for applications in microbial or mammalian cell culture, as well as other metals used for applications in chemical process industries, such as catalyst separation and recycle. In certain embodiments, the settler devices of this disclosure include stainless steel surfaces that are partially or completely electropolished to provide smooth surfaces that cells or particles may slide down after settling out of liquid suspension. In certain embodiments, some or all of the surfaces of the settler device may be coated with a non-sticky plastic or silicone, such as dimethyldichlorosilane. In related embodiments, the material construction of any of these settler devices may be non-metals, including plastics, for use in, for example, single use disposable bioreactor bags, etc. While metal settling devices of the invention can be constructed via standard plate rolling and welding of steel angular plates to the bottom of the spiral plate, a plastic settler device of this disclosure, or individual parts thereof, may be more easily fabricated continuously as a single piece using, for example, injection molding or three-dimensional printing technologies.

In each of the embodiments of this disclosure, liquid may be directed into, or drawn out of, any of the ports or openings in the conical settling device by one or more pumps (for example a peristaltic pump) in liquid communication with the port or opening. Such pumps, or other means causing the liquid to flow into or out of the settler devices, may operate continuously or intermittently. If operated intermittently, during the period when the pump is off, settling of particles or cells occurs while the surrounding fluid is still. This allows those particles or cells that have already settled to slide down the inclined conical surfaces unhindered by the upward flow of liquid. Intermittent operation has the advantage that it can improve the speed at which the cells slide downwardly, thereby improving cell viability and productivity. In a specific embodiment, a pump is used to direct a liquid suspension of cells from a bioreactor or fermentation media into the settler devices of the present disclosure.

In each of the embodiments of this disclosure, the top plate, or lid, covering the cyclone housing may be concave, rising to a central core point. In these embodiments, the angle of rise in the concave top plate may preferably be between 1 degree and 10 degrees, more preferably between 1 degree and 5 degrees. This concave top plate creates a tent-like space above the center of the cyclone housing. Gas, bubbles, froth or the like may accumulate in this space and a tube may be inserted through an opening in the cyclone housing or through an opening in the top plate to withdraw such gasses, etc. from the space beneath the top of the cyclone housing. Similarly, fluid or gas may be pumped into the cyclone housing through such tube that is inserted through an opening in the cyclone housing or through an opening in the top plate.

### Methods of Use and Operation Processes

The settling devices of this disclosure have applications in numerous fields, including (i) high cell density biological (mammalian, microbial, plant or algal) cell cultures secreting polypeptides, hormones, proteins or glycoproteins, sub-unit vaccines, viruses, virus-like particles or other small chemical products, such as ethanol, isobutanol, isoprenoids, etc., (ii) separating and recycling porous or non-porous solid catalyst particles catalyzing chemical reactions in liquid or gas phase surrounding solid particles, (iii) separating and collecting newly formed solids in physical transformations such as crystallization, flocculation, agglomeration, precipitation, etc., from the surround liquid phase, and (iv) clarifying process water in large scale municipal or commercial waste water treatment plants by settling and removing complex biological consortia or activated sludge or other solid particles.

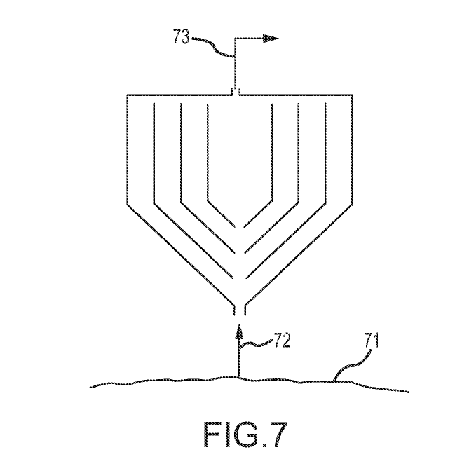

FIG. 5 shows an effective flow pattern of liquid and particles through a settler device of this disclosure, producing maximal particle separation efficiency. As depicted in FIG. 5, a particle containing liquid (including, for example, cell culture liquid, waste water or reaction fluid containing solid catalyst particles, etc.) is preferably introduced tangentially into a settler device of this disclosure near the top of the cylindrical housing from the side along the direction of arrow 51, to take full advantage of the centrifugal forces on the particles, pushing them against the wall of the spiral vertical plate. The channel within the spiral vertical plate creates increased contact area, residence time and gradually increasing centrifugal force for the particles to be pushed against the spiral wall. The particles or cells sliding down the walls and settling in the vertical sedimentation columns of the spiral channel enter an enhanced sedimentation zone of the conical surfaces. Particles or cells settled on the inclined conical surfaces are swept down to the opening at the bottom of the conical housing by the dense liquid (i.e. liquid containing concentrated particles or cells) exiting at the bottom of the cone in the direction of arrow 53. Liquid exiting the outlet in the direction of arrow 53 contains concentrated cells or particles to be recycled to a bioreactor or directed to a chemical reactor, or waste water tank, etc. Clarified liquid containing any secreted proteins or other products and smaller particles or dead cells or cell debris, is harvested at an outlet along the direction of arrow 52.

In one embodiment, clarified liquid entering the central tube is removed or harvested at the top by suction from a pump attached on the tube connected to the top port. The dense liquid containing concentrated particles or cells can be recycled to the reactor or bioreactor or harvested as desired. The flow rate of the dense liquid exiting the bottom of the conical device is ideally equal to the difference in the inlet flow rate at the tangential entry near the top and outlet flow rate at the top, each controlled by a separate pump. Additional control valves may be added to the bottom liquid exit tube to ensure that the clarified liquid exits at the top and may be fully opened as needed to prevent or remove any dense packing of particles clogging the underflow stream.

Another flow configuration for liquid and particles through a settler device of this disclosure is depicted in FIG. 6. This flow configuration results in a slightly reduced separation efficiency compared to the flow configuration depicted in FIG. 5 because the top vertical entry does not take advantage of any small centrifugal forces which can be created by the tangential entry depicted in FIG. 5. Nevertheless, this configuration makes use of the major separating principle of enhanced sedimentation on inclined surfaces and will be sufficient for full separation of larger live mammalian cells from smaller dead cells and cell debris if the device is sized adequately for use with a bioreactor. In this operating embodiment, liquid containing cells or solid particles, or waste water is directed into the top of the settler device along the direction of arrow 61. Outlet liquid outlet containing concentrated cells, particles or sludge to be recycled back to the bioreactor, chemical reactor or waste water tank exits the settler device along the direction of arrow 62. Clarified liquid containing any secreted proteins, smaller dead cells or cell debris, is harvested from the settler device near the top of the conical housing proximate the top of the settler device, along the direction of arrow 63.

A third flow configuration useful for a settler device of this disclosure that includes only two ports is depicted in FIG. 7. A liquid suspension is direction along the direction of arrow 72 from a single-use disposable plastic bioreactor bag (71), which may be culturing either mammalian or microbial cells secreting one or more desired chemical products, into a bottom port of the settler device. The inlet port is firmly attached to the plastic bioreactor bag (71), but without any pump. This inlet port carries both the contents of the bioreactor bag upwards, and the settled cells downward back to the bioreactor bag. Thus, the feed inlet to the settler device and the underflow of settled particles or cells cross paths in the same bottom port of the conical settler device, i.e., the two streams (feed inlet and underflow) occur via the same bottom port. This flow configuration may be useful in connection with a single use, plastic disposable bioreactor bag, or with other applications used with smaller scale settler devices of this disclosure. Such smaller scale settler devices are typically made of plastic, and may be single-use, disposable plastic devices. In this flow configuration, clarified liquid outlet containing any secreted protein product and fewer smaller cells or cell debris, exits from the top port of the settler device along the direction of arrow 73.

If a third port is provided in the configuration of FIG. 7, it may be used initially to provide a vacuum suction to fill up or prime the device. In some embodiments, the third port is not provided in the settler device as it is not needed in conjunction with this embodiment containing a single port in which feed inlet and underflow of settled particles or cells cross paths in the same bottom port.

For the smaller scale applications with a plastic bag bioreactor with only two vertical ports used in the flow configuration as shown in FIG. 7, it is advantageous to extend the conical spiral surface closer to the center of the settler device to prevent a direct flow of inlet cell culture broth from the bottom port up through the central opening in the device. One possible extension of the conical spiral surface to ensure the flow of cell culture broth from bottom inlet through all the spiral conical and vertical sedimentation chambers of the device is shown in the sectional diagram of FIG. 3.

One parameter that may be adjusted in these methods of using the settler devices of this disclosure is the liquid flow rate into and out of the settler devices. The liquid flow rate will depend entirely on the particular application of the device and the rate can be varied in order to protect the particles being settled and separated from the clarified liquid. Specifically, the flow rate may need to be adjusted to protect the viability of living cells that may be separated in the settler devices of this disclosure and returned to a cell culture, but the flow rate should also be adjusted to prevent substantial cell or particle build up in the settler devices or clogging of the conduits that transfer liquid into and out of the settler devices.

Each publication or patent cited herein is incorporated herein by reference in its entirety. The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention.

### EXAMPLES

### Example 1

### Yeast or Other Microbial Cells Secreting Protein Products

Recombinant microbial cells such as yeast or fungal (Pichia pastoris, Saccharomyces cerevisiae, Kluyveromyces lactis, Aspergillus niger, etc.) or bacterial (Escherichia coli, Bacillus subtilis, etc.) cells, which have been engineered to secrete heterologous proteins or naturally secreting enzymes (e.g. A. niger, B. subtilis, etc.) can be grown in bioreactors attached to settler devices of the present disclosure to recycle live and productive cells back to the bioreactor, which will thereby achieve high cell densities and high productivities. Fresh nutrient media is continuously supplied to the live and productive cells inside the high cell density bioreactors and the secreted proteins or enzymes are continuously harvested in the clarified outlet from the top or top-side outlets as shown in FIGS. 5, 6 and 7, while the concentrated live and productive cells are returned back to the bioreactor. As dead cells and a small fraction of live cells are continuously removed from the bioreactor via the harvest outlet, cell growth and protein production can be maintained indefinitely, without the need to terminate the bioreactor operation. In our operations with yeast Pichia cells, we have operated this perfusion bioreactor for over a month. As the microbial cells grow in suspension culture and the cell retention device can be scaled up to any desired size, the present invention can be attached to suspension bioreactors of sizes varying from lab scale (<1 liter) to industrial scale (>50,000 liters) to achieve high cell density perfusion cultures.

### Example 2

### Removing Yeast Cells From Beer

In large-scale brewing operations, yeast cells are removed from the product beer by filtration devices, which regularly get clogged, or centrifugation devices, which are expensive high-speed mechanical devices. These devices can be readily replaced by the present invention to clarify beer from the top outlets and remove the concentrated yeast cell suspension from the bottom outlet. Hydrocyclones were unsuccessfully tested for exactly this application (Yuan et al., 1996; Cilliers and Harrison, 1997). Due to the increased residence time in the spiral channels and enhanced sedimentation in the conical spiral settler zone of the present invention, we have achieved successful separation of yeast cells from cell culture liquid, harvesting the culture supernatant containing only about 5% of the cells entering the settler device in its first operation. As the device can be scaled up or down to increase or decrease its cell separation efficiency, it is feasible to obtain completely cell-free beer from the harvest port of a settler device of this disclosure.

### Example 3

### Mammalian Cell Perfusion Cultures

Enhanced sedimentation of recombinant mammalian cells and murine hybridoma cells in inclined settlers have already been demonstrated successfully (Batt et al., 1990 and Searles et al., 1994) and scaled up in lamellar settlers (Thompson and Wilson, U.S. Pat. No. 5,817,505, 1998). While the lamellar settlers are scaled up in three dimensions independently, the present invention of a conical spiral settler device can be scaled up in three dimensions simultaneously by simply increasing its radius, as discussed above. Further, the present invention benefits from an additional cell separating mechanism of increasing centrifugal forces as the cell culture liquid passes through the decreasing radius of the vertical spiral section, followed by the enhanced sedimentation in the conical spiral settling zone of the settler devices of this disclosure. Thus, the settler devices of the present disclosure is a more compact and more easily scalable cell retention device with proven applications in mammalian cell cultures secreting glycoproteins, such as monoclonal antibodies and other therapeutic proteins, including sub-unit vaccines. The clarified harvest output from the liquid outlets (FIGS. 5, 6 and 7) containing the secreted protein is harvested continuously from the cell retention device, while the concentrated cells from the bottom outlet are recycled back to the bioreactor, resulting in a high cell density perfusion bioreactor that can be operated indefinitely, i.e. over several months of continuous perfusion operation. The continuous high titer harvest from a single, 1000-liter, high cell density perfusion bioreactor can be more than the accumulated production from a large (>20,000 liter) fed-batch bioreactor on an annual basis.

### Example 4

### Vaccines, Viruses or Virus-Like Particles Production

Production of vaccines, such as viruses or virus-like particles (VLPs), is usually carried out by infection and lysis of live mammalian or insect cells in a batch or fed-batch bioreactor culture. Viruses or virus-like particles are released from the infected cell in a lytic process after large intracellular production of these viruses or virus-like particles. With the large difference in the size (sub-micron or nanometer scale) of these particles compared to the size (about 5-20 microns) of live mammalian and insect cells, the separation of the viruses or virus-like particles from the bioreactor culture is very simple. By controlling the harvest or outlet rate of cell culture broth containing mostly viruses or VLPs, along with cell debris, it is possible to retain a smaller number of the infective particles inside the bioreactor along with the growing live cells to continually infect and produce vaccines in a continuous perfusion bioreactor attached to a settler device of this disclosure for continuous harvest of viruses and VLPs.

### Example 5

### Solid Catalyst Particle Separation and Recycle

Separation of solid catalyst particles for recycle into a chemical reactor and reuse in further catalyzing liquid phase chemical reactions, such as Fischer-Tropsch synthesis, has been previously demonstrated with lamellar settlers (U.S. Pat. No. 6,720,358, 2001). Many such two-phase chemical reactions, involving solid catalyst particles in liquid or gas phase reactions can be enhanced by the use of the settler devices of the present invention, which provide a more compact particle separation device to accomplish the same solids separation and recycle as demonstrated with lamellar settlers.

### Example 6

### Plant and Algal Cell Harvesting

Recombinant plant cell cultures secreting valuable products, while not yet commercially viable, are yet another field of potential applications for the settling devices of the present invention. Inclined settlers have been used in several plant cell culture applications. Such devices can be replaced by the more compact conical spiral settler devices of present disclosure. With the size of plant cells being much larger than those of yeast or mammalian cells, the cell separation efficiency will be much higher with single plant cells or plant tissue cultures.

A more immediate application of devices of this disclosure may be found in the harvesting of algal cells from large scale cultivation ponds to harvest biodiesel products from inside algal cells. Relatively dilute algal cell mass in large (acre sized) shallow ponds converting solar energy into intracellular fat or fatty acid storage can be harvested easily through the settler devices of this disclosure and the concentrated algal cells can be harvested from the bottom outlet of these conical settler devices.

### Example 7

### Municipal Waste Water Treatment

Large scale municipal waste water treatment plants (using activated sludge or consortia of multiple bacterial species for degradation of biological and organic waste in sewage or waste water) commonly use large settling tanks and more modern versions of these plants use lamellar settlers to remove the clarified water from the sludge. The conical spiral settler devices of this disclosure can be scaled up to the larger sizes required in these plants, while remaining smaller in size than the large settling tanks or lamellar settlers currently used in these treatment plants.

### Example 8

### Industrial Process Water Clarification

Large scale water treatment plants, cleaning either industrial waste water or natural sources of turbid water containing suspended solids, use large scale settling tanks or lamellar inclined settlers. These large scale devices can now be replaced with the more compact conical spiral settler devices of this disclosure to accomplish the same goal of clarifying water for industrial reuse or municipal supply of fresh water.

What is claimed is:

1. A particle settling device comprising:
a. a cyclone housing;
b. a spiral vertical plate disposed inside the cyclone housing, the spiral vertical plate joined at one end with a spiral conical surface tapering down to a first opening in the cyclone housing; and,
c. at least one additional opening in the cyclone housing opposite the first opening.

2. The device of claim 1, wherein the spiral vertical plates comprise at least one material selected from the group consisting of a metal and a plastic.

3. The device of claim 1, wherein the spiral vertical plates comprise stainless steel.

4. The device of claim 1, composed entirely of stainless steel.

5. The device of claim 1, composed entirely of plastic.

6. The device of any one of claims 2-4, wherein the spiral vertical plate and the spiral conical surface are metals joined by welding.

7. The device of any one of claims 1-6, wherein the spiral vertical plate is arranged in a substantially vertical position within the cyclone housing and an angle of inclination for the conical spiral surface is between about 30 degrees and about 60 degrees from vertical.

8. The device of claim 7, wherein the angle of inclination for the conical spiral surface is about 30 degrees from vertical.

9. The device of claim 7, wherein the angle of inclination for the conical spiral surface is about 60 degrees from vertical.

10. The device of claim 1, wherein at least one surface of the cyclone housing, the spiral vertical plate or the spiral conical surface is coated with a non-sticky plastic or silicone.

11. The device of claim 1, wherein a width of a channel formed between adjacent surfaces of the spiral vertical plate is between about 1 mm and about 50 mm.

12. The device of claim 1, wherein a number of spirals forming adjacent surfaces of the spiral vertical plate is between about 3 and about 30.

13. The device of claim 1, wherein the first opening is configured as an outlet in liquid communication with the outside and the inside of the cyclone housing substantially opposite the spiral vertical plate.

14. The device of claim 13, further comprising a closure over at least a portion of the cyclone housing at an end of the cyclone housing adjacent the spiral vertical plate and opposite the first opening.

15. The device of claim 14, wherein the at least one additional opening in the cyclone housing is configured to open from a side of the cyclone housing tangential to the spiral vertical plate, in liquid communication with the outside and the inside of the cyclone housing.

16. The device of claim 15, further comprising a liquid harvest outlet formed in the closure, in liquid communication with the outside and the inside of the cyclone housing.

17. The device of claim 14, wherein the at least one additional opening in the cyclone housing is configured to open into the closure, in liquid communication with the outside and the inside of the cyclone housing.

18. The device of claim 17, further comprising a liquid harvest outlet formed in a side of the cyclone housing tangential to the spiral vertical plate, in liquid communication with the outside and the inside of the cyclone housing.

19. The device of claim 14, wherein the first opening is in liquid communication with the outside and the inside of the cyclone housing and is configured to communicate liquid both into and out of the cyclone housing.

20. A particle settling device comprising:
a. a cyclone housing;
b. a spiral vertical plate disposed inside the cyclone housing opposite a first opening in the cyclone housing, and,
c. at least one additional opening in the cyclone housing opposite the first opening.

21. The device of claim 20, further comprising a spiral conical surface joined to one end of the spiral vertical plate, the spiral conical surface tapering down to the first opening in the cyclone housing;

22. The device of claim 20, wherein the spiral vertical plates comprise at least one material selected from the group consisting of a metal and a plastic.

23. The device of claim 20, wherein the spiral vertical plates comprise stainless steel.

24. The device of claim 20, composed entirely of stainless steel.

25. The device of claim 20, composed entirely of plastic.

26. The device of any one of claims 20-25, wherein the spiral vertical plate is arranged in a substantially vertical position within the cyclone housing.

27. The device of claim 20, wherein at least one surface of the cyclone housing, the spiral vertical plate or the spiral conical surface is coated with a non-sticky plastic or silicone.

28. The device of claim 20, wherein a width of a channel formed between adjacent surfaces of the spiral vertical plate is between about 1 mm and about 50 mm.

29. The device of claim 20, wherein a number of spirals forming adjacent surfaces of the spiral vertical plate is between about 3 and about 30.

30. The device of claim 20, wherein the first opening is configured as an outlet in liquid communication with the outside and the inside of the cyclone housing substantially opposite the spiral vertical plate.

31. The device of claim 30, further comprising a closure over at least a portion of the cyclone housing at an end of the cyclone housing adjacent the spiral vertical plate and opposite the first opening.

32. The device of claim 20, wherein the at least one additional opening in the cyclone housing is configured to open from a side of the cyclone housing tangential to the spiral vertical plate, in liquid communication with the outside and the inside of the cyclone housing.

33. The device of claim 32, further comprising a liquid harvest outlet formed in the closure, in liquid communication with the outside and the inside of the cyclone housing.

34. The device of claim 32, wherein the at least one additional opening in the cyclone housing is configured to open into the closure, in liquid communication with the outside and the inside of the cyclone housing.

35. The device of claim 32, further comprising a liquid harvest outlet formed in a side of the cyclone housing tangential to the spiral vertical plate, in liquid communication with the outside and the inside of the cyclone housing.

36. The device of claim 32, wherein the first opening is in liquid communication with the outside and the inside of the cyclone housing and is configured to communicate liquid both into and out of the cyclone housing.

37. A particle settling device comprising:
a. a cyclone housing;
b. a spiral vertical plate disposed inside the cyclone housing, wherein a height of the spiral vertical plate decreases from the exterior to the interior of the spiral;
c. a spiral conical surface joined at one end to the spiral vertical plate tapering down to a first opening in the cyclone housing; and,
d. at least one additional opening in the cyclone housing opposite the first opening.

38. The device of claim 37, composed entirely of a plastic.

39. The device of claim 37, wherein the spiral vertical plate is arranged in a substantially vertical position within the cyclone housing and an angle of inclination for the conical spiral surface is between about 30 degrees and about 60 degrees from vertical.

40. The device of claim 39, wherein the angle of inclination for the conical spiral surface is about 30 degrees from vertical.

41. The device of claim 39, wherein the angle of inclination for the conical spiral surface is about 60 degrees from vertical.

42. The device of claim 37, wherein at least one surface of the cyclone housing, the spiral vertical plate or the spiral conical surface is coated with a non-sticky plastic or silicone.

43. The device of claim 37, wherein a width of a channel formed between adjacent surfaces of the spiral vertical plate is between about 1 mm and about 50 mm.

44. The device of claim 37, wherein a number of spirals forming adjacent surfaces of the spiral vertical plate is between about 3 and about 30.

45. The device of claim 37, wherein the first opening is configured as an outlet in liquid communication with the outside and the inside of the cyclone housing substantially opposite the spiral vertical plate.

46. The device of claim 45, further comprising a closure over at least a portion of the cyclone housing at an end of the cyclone housing adjacent the spiral vertical plate and opposite the first opening.

47. The device of claim 37, wherein the at least one additional opening in the cyclone housing is configured to open from a side of the cyclone housing tangential to the spiral vertical plate, in liquid communication with the outside and the inside of the cyclone housing.

48. The device of claim 47, further comprising a liquid harvest outlet formed in the closure, in liquid communication with the outside and the inside of the cyclone housing.

49. The device of claim 48, further comprising a liquid harvest outlet formed in a side of the cyclone housing tangential to the spiral vertical plate, in liquid communication with the outside and the inside of the cyclone housing.

50. The device of claim 37, wherein the first opening is in liquid communication with the outside and the inside of the cyclone housing and is configured to communicate liquid both into and out of the cyclone housing.

51. A particle settling device comprising:
a. a cyclone housing;
b. at least two conical plates disposed inside the cyclone housing, the at least two conical plates stacked one above the other, each of the at least two conical plates comprising at least three vertical supports that project from a surface of each conical plate to hold each conical plate at a substantially constant distance from a next conical plate in the stack of at least two conical plates, each of the conical plates comprising an opening sufficient to allow liquid to pass through the openings in the conical plates, the stack of at least two conical plates tapering down to a first opening in the cyclone housing; and,
c. at least one additional opening in the cyclone housing opposite the first opening.

52. The device of claim 51, wherein the at least two conical plates comprise at least one material selected from the group consisting of a metal and a plastic.

53. The device of claim 51, wherein the at least two conical plates comprise stainless steel.

54. The device of claim 51, composed entirely of stainless steel.

55. The device of claim 51, composed entirely of plastic.

56. The device of any one of claims 52-54, wherein the spiral vertical plate and the spiral conical surface are metals joined by welding.

57. The device of any one of claims 51-56, wherein the stack of at least two conical plates is arranged in a substantially vertical position within the cyclone housing and an angle of inclination for a surface of each of the at least two conical plates is between about 30 degrees and about 60 degrees from vertical.

58. The device of claim 57, wherein the angle of inclination for the surface is about 30 degrees from vertical.

59. The device of claim 57, wherein the angle of inclination for the surface is about 60 degrees from vertical.

60. The device of claim 51, wherein at least one surface of the cyclone housing, and the at least two conical plates is coated with a non-sticky plastic or silicone.

61. The device of claim 1, wherein a width of a channel formed between adjacent surfaces of the at least two conical plates is between about 1 mm and about 50 mm.

62. The device of claim 1, wherein a number of conical plates in the stack of at least two conical plates is between about 3 and about 30 conical plates.

63. The device of claim 51, wherein the first opening is configured as an outlet in liquid communication with the outside and the inside of the cyclone housing.

64. The device of claim 51, further comprising a closure over at least a portion of the cyclone housing at an end of the cyclone housing opposite the first opening.

65. The device of claim 64, further comprising a liquid harvest outlet formed in the closure, in liquid communication with the outside and the inside of the cyclone housing.

66. The device of claim 65, further comprising at least one additional opening in the cyclone housing configured to open into the closure, in liquid communication with the outside and the inside of the cyclone housing.

67. The device of claim 51, further comprising at least one additional opening in the cyclone housing configured to open from a side of the cyclone housing tangential to the at least two conical plates, in liquid communication with the outside and the inside of the cyclone housing.

68. The device of claim 51, further comprising a liquid harvest outlet formed in a side of the cyclone housing tangential to the at least two conical plates, in liquid communication with the outside and the inside of the cyclone housing.

69. The device of claim 51, wherein the first opening is in liquid communication with the outside and the inside of the cyclone housing and is configured to communicate liquid both into and out of the cyclone housing.

70. A method of settling particles in a liquid suspension, comprising:
a. introducing a liquid suspension into a particle settling device comprising:
i. a cyclone housing;
ii. a spiral vertical plate disposed inside the cyclone housing, the spiral vertical plate joined at one end with a spiral conical surface tapering down to a first opening in the cyclone housing; and,
iii. at least one additional opening in the cyclone housing opposite the first opening configured to receive the liquid suspension,
b. collecting particles from the first opening; and,
c. collecting a liquid from the settling device.

71. The method of claim 70, wherein the particle settling device further comprises a closure over at least a portion of the cyclone housing at an end of the cyclone housing adjacent the spiral vertical plate and opposite the first opening, and liquid is collected from the particle settling device from at least one opening formed in the closure.

72. The method of claim 70, wherein the at least one additional opening in the cyclone housing is configured to open from a side of the cyclone housing tangential to the spiral vertical plate, in liquid communication with the outside and the inside of the cyclone housing.

73. The method of claim 70, wherein the liquid suspension comprises at least one of a recombinant cell suspension, an alcoholic fermentation, a suspension of solid catalyst particles, a municipal waste water, industrial waste water.

74. The method of claim 70, wherein the liquid suspension comprises at least one of mammalian cells, bacterial cells, yeast cells, plant cells, insect cells.

75. The method of claim 70, wherein the liquid suspension comprises at least one of biodiesel-producing algae cells, mammalian and/or murine hybridoma cells, and yeast in beer.

76. The method of claim 70, wherein the liquid suspension comprises recombinant microbial cells selected from Pichia pastoris, Saccharomyces cerevisiae, Kluyveromyces lactis, Aspergillus niger, Escherichia coli, and Bacillus subtilis.

77. The method of claim 70, wherein the step of introducing a liquid suspension comprises directing a liquid suspension from a plastic disposable bioreactor bag into the particle settling device.

78. The method of claim 70, wherein the liquid collected comprises at least one of biological molecules, organic or inorganic compounds, chemical reactants, and chemical reaction products.

79. The method of claim 70, wherein the liquid collected comprises at least one of hydrocarbons, polypeptides, proteins, alcohols, fatty acids, hormones, carbohydrates, antibodies, isoprenoids, biodiesel, and beer.

80. The method of claim 70, wherein the liquid collected comprises at least one of insulin or its analogs, monoclonal antibodies, growth factors, sub-unit vaccines, viruses, virus-like particles, colony stimulating factors and erythropoietin (EPO).
